(19) United States
(12) Patent Application Publication
(10) Pub. No.: US 2026/0191411 A1
(43) Pub. Date: Jul. 9, 2026

(54) METHOD AND IMAGING DEVICE FOR IN VIVO DETECTION OF AUTOFLUORESCENCE OF AN OCULAR FUNDUS

(71) Applicant: RHEINISCHE FRIEDRICH-WILHELMS-UNIVERSITÄT BONN, Bonn (DE)

(72) Inventor: Thomas ACH, BONN (DE)

(21) Appl. No.: 19/131,097

(22) PCT Filed: Nov. 29, 2023
(86) PCT No.: PCT/EP2023/083446

§ 371 Date: May 19, 2025

(30) Foreign Application Priority Data
Nov. 30, 2022 (DE) 10 2022 131 803.6

(51) Int. Cl. A61B 3/12; A61B 3/113

(52) U.S. Cl. CPC A61B 3/1241; A61B 3/113

(57) ABSTRACT

A method and a spectral imaging device for in vivo detection of autofluorescence of an ocular fundus of an eye includes providing a light signal with a predetermined wavelength in the short-wave range, detecting a first eye position and dividing the ocular fundus into a grid comprising a multiplicity of pixels, determining an area of the ocular fundus to be examined, wherein the area is determined by a selection of a plurality of pixels, exciting the ocular fundus of a pixel of the area to be examined using the light signal, detecting a further ocular position differing from the first ocular position and tracking the grid in accordance with the detected further ocular position, detecting at least one emission signal emitted by the pixel of the ocular fundus, supplying the emission signal to an evaluation unit, and repeating the previous four steps for each pixel of the plurality of pixels.

The invention relates to a method for in vivo detection of autofluorescence of an ocular fundus of an eye.

Age-related macular degeneration (AMD) is the most common cause of blindness in developed countries. In Germany alone, over 5 million people are affected. So far, only the late, advanced form of AMD can be treated. There is no cure and, in particular, no therapeutic intervention for the early and intermediate forms. The early and intermediate forms of AMD are defined, among other things, by the occurrence of drusen and changes in the retinal pigment epithelium. As precursors, deposits in the area of the basal lamina of the retinal pigment epithelium (RPE), basal laminar deposits (BLamD), as well as between the basal lamina of the RPE and the inner collagenous layer of the Bruch membrane, basal linear deposits (BLinD), are found in normal aging and in AMD.

In order to improve diagnosis and therapy, early detection of changes or deposits even before clinical evidence of drusen is essential. No methods for achieving this are known in the prior art.

On this basis, the object of the invention is to be able to detect and precisely localise changes preceding drusen (BLamD and BLinD) in vivo at an early stage.

This object is solved by the subject of patent claim 1. Preferred embodiments can be found in the sub-claims.

According to the invention, a method for in vivo detection of autofluorescence of an ocular fundus of an eye is provided, comprising the following method steps:
a) Providing a light signal with a predetermined wavelength in the short-wave range,
b) Detecting a first eye position and dividing the ocular fundus into a grid comprising a multiplicity of pixels depending on the detected first eye position,
c) Determining an area of the ocular fundus to be examined, wherein the area of the ocular fundus to be examined is determined by a selection of a plurality of pixels,
d1) Exciting the ocular fundus of a pixel in the region to be examined by means of the light signal,
d2) Detecting a further ocular position differing from the first ocular position and tracking the grid in accordance with the detected further ocular position,
e) Detecting at least one emission signal emitted by the pixel of the ocular fundus,
f) Supplying the emission signal to an evaluation unit,
g) Repeating steps d1) to f) for each pixel of the area to be examined.

In the present case, the ‘ocular fundus’ refers to the area behind the transparent vitreous humour on the inner wall of the eyeball. The ocular fundus includes, in particular, the retina, the retinal pigment epithelium (RPE), the basal membrane, the Bruch's membrane and the choriocapillaris.

‘In vivo’ refers to an examination of the ocular fundus in a living patient or organism.

‘Tracking’ refers to the correction of the eye position during the examination. The movements of the eye are recorded and taken into account during the recording of emission signals. In particular, tracking is carried out using a tracking system that is operated in constant feedback with a scanning unit to record the emission signals.

It has been shown that, after the ocular fundus has been stimulated with a certain light, these deposits or precursors of the drusen exhibit auto-fluorescence properties and can be microscopically visualised as flat sub-RPE deposits using an emission signal.

It is thus a key point of the invention that the method not only detects deposits or precursors of the drusen, but can also localise them precisely on the living eye, even when the position of the eye is constantly changing. The eye or the ocular fundus is subdivided by means of a grid. The grid is also tracked when the eye moves and the eye position changes, so that scanning of the ocular fundus is possible along the same pixel even when the eye position changes.

Preferably, the excitation and measurement per pixel is repeated several times so that an overall averaged emission signal can be generated. By repeating the excitation and scanning while constantly tracking the position of the eye, several emission signals can be recorded per pixel and combined into an average emission signal. This minimises uncertainties and increases the overall quality of the measurement.

According to a preferred embodiment of the invention, the method comprises the following further steps:
h) Determining whether a deposit is present on the fundus using the emission signal, and
i) Assigning the deposit to a pixel.

The deposits exhibit autofluorescent properties when excited with a specific light. The emission signals can be examined using appropriate evaluation algorithms. The emission signals characteristic of the precursors of drusen can be detected and assigned to individual pixels. In this way, the deposits typical of the precursors at the ocular fundus can be detected and localised on the basis of the emission signals.

According to a preferred embodiment of the invention, the predetermined wavelength can be selected from a range of between 350 nm and 750 nm, preferably between 360 nm and 500 nm. In principle, the method can also be used to detect the drusen themselves, in addition to the preliminary stages of drusen. Different wavelengths are required for the occurrence of autofluorescence in drusen and in the preliminary stages of drusen. The emission signals characteristic of drusen and preliminary stages of drusen can be detected and assigned to individual pixels. This requires a light signal with a correspondingly broad electromagnetic spectrum, preferably in the blue or deep blue to violet wavelength range. In this way, the emission signals can be used to detect and localise not only drusen but also the deposits typical of the preliminary stages at the ocular fundus.

According to a preferred embodiment of the invention, the predetermined wavelength can be continuously adjusted. This makes it possible to excite the ocular fundus continuously. In this way, autofluorescent structures can be excited particularly efficiently and simply with different excitation wavelengths, since the excitation wavelength can be matched to the structure to be examined.

According to the invention, a spectral imaging device is also provided for in vivo detection of autofluorescence of an ocular fundus, comprising
a light source for generating a light signal with a predetermined wavelength in the short-wave range for exciting at least one region of the ocular fundus,
a position correction unit with a tracking system for detecting and tracking an eye position,
a scanning unit for directing the light signal and scanning at least a region of the ocular fundus depending on the eye position, and
a sensor for detecting an emission signal emitted by the ocular fundus depending on the eye position.

The light source is preferably a laser source. The laser source generates a laser beam with a predetermined wavelength.

In the present case, a position correction unit is understood to be, in particular, a unit that can detect the position of the eye and correct it by means of the tracking system. In this way, eye movements can be tracked and taken into account when scanning the ocular fundus. The position correction unit and the tracking system are realised in particular by means of software that synchronises image recordings of the ocular fundus according to the deviation caused by eye movements.

The sensor for detecting an emission signal emitted from the ocular fundus comprises in particular a camera that scans the ocular fundus pixel by pixel. Preferably, the sensor comprises in particular a sensor that is designed for optical coherence tomography (OCT). With this type of sensor, the ocular fundus can be scanned pixel by pixel. Alternatively, the camera comprises a snapshot camera. A snapshot camera is a camera that can take a large number of snapshots. In this way, existing OCT systems or low-cost snapshot cameras can also be used for the imaging device according to the invention.

According to a preferred embodiment of the invention, the light source is designed to generate a light signal with a predetermined wavelength in a range of between 350 nm and 750 nm, preferably between 360 nm and 500 nm. In this way, the spectral imaging device can be used to detect several different changes in the tissue, since the light source can generate different wavelengths to excite different structures.

According to a preferred embodiment of the invention, the scanning unit comprises a plurality of deformable mirrors. The deformable mirrors perform the function of a scanning laser unit, since they can be controlled and modified in such a way that they can be precisely aligned. In this way, the ocular fundus can be scanned pixel by pixel using the light signal and the emission signals can be detected.

According to a preferred embodiment of the invention, the spectral imaging device also comprises an evaluation unit for determining, on the basis of the emission signal, whether a deposit is present on the ocular fundus and for localising the deposit based on the position of the eye. The evaluation unit analyses the detected emission signals and filters out the emission signals that indicate a deposit or a preliminary stage of the drusen. These emission signals can then be assigned locally. Preferably, the emission signals can be post-processed using the evaluation unit. The post-processing includes in particular the decomposition of the emission signals and/or the dimensional reduction of the data using a non-negative matrix factorisation. For this purpose, a matrix with entries in the non-negative real numbers is linearly decomposed into factors of rank 1. Special algorithms can be used to find a decomposition in which the individual factors are also non-negative. In many cases, this requirement leads to decompositions that are easier to interpret and represent the data as the sum of clearly separated components. Preferably, the post-processing of the data for detecting signals in the wavelength range of less than 450 nm is optimised.

According to a preferred embodiment of the invention, the light source has an excitation filter for filtering the light signal. Further preferably, the sensor comprises an emission filter for filtering the emission signal. In this way, the light signal and/or the emission signal can be limited to the wavelength ranges relevant for the examination, so that unavoidable wavelengths outside the relevant spectrum can be filtered out particularly easily.

The invention is further explained in detail below with reference to a preferred embodiment of the invention with reference to the drawings.

In the drawings,

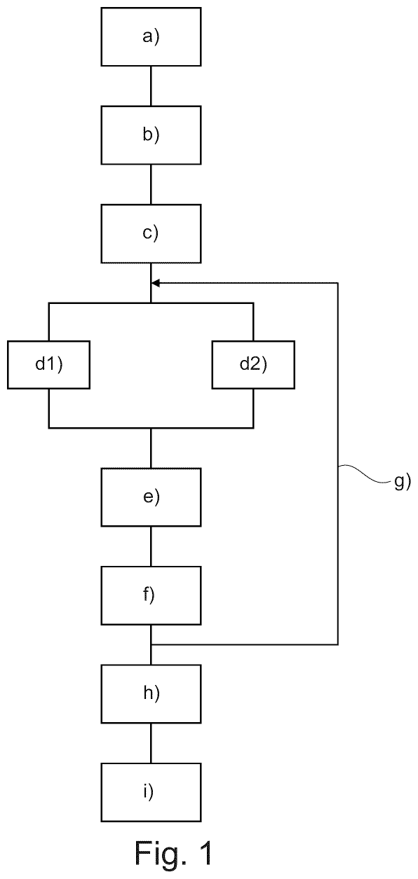

FIG. 1 schematically shows a method for in vivo detection of autofluorescence of an ocular fundus according to a preferred embodiment of the invention,

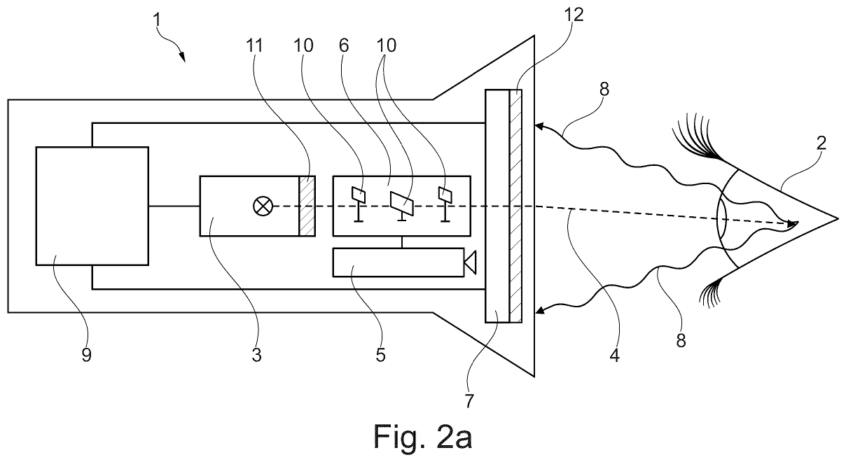

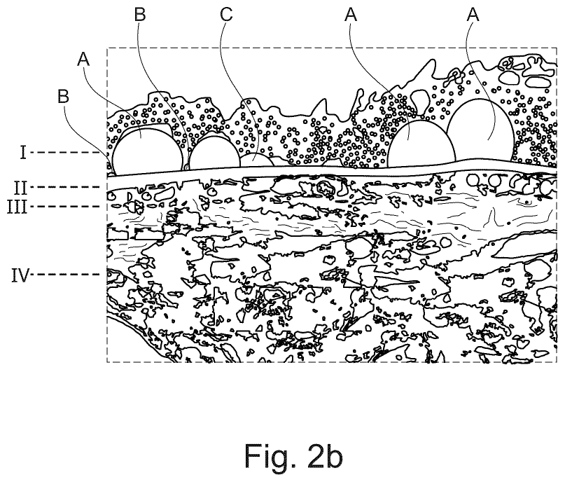

FIG. 2a schematically shows a spectral imaging device according to a preferred embodiment of the invention,

FIG. 2b schematically shows a histological representation of deposits in an ocular fundus in a vertical sectional view.

FIG. 1 schematically shows a method for in vivo detection of autofluorescence of an ocular fundus. In a first step a) a light signal 4 is provided to excite the ocular fundus. The light signal 4 covers a wavelength of between 350 nm and 750 nm, preferably between 360 nm and 500 nm, and can be continuously adjusted in this range. In a second step b) the eye to be examined is measured. This means that an initial eye position is determined. The ocular fundus in this eye position is subdivided into several pixels by dividing the ocular fundus into a grid-like pattern. After that, an area to be examined is determined by selecting several pixels that form this area.

Then, in steps d1) and d2), for one pixel of the area to be examined, the ocular fundus of this pixel is targeted and stimulated with the light signal 4. At the same time, the position of the eye is permanently monitored and the virtual grid is adjusted according to the current position of the eye. In this way, individual pixels are specifically targeted without moving due to movements of the eye. In a subsequent step e), the emission signals 8 emitted by the ocular fundus are detected and then f) made available to an evaluation unit 9.

The scanning, i.e. the targeted control of individual pixels with simultaneous tracking of the eye position, is repeated for each pixel of the area to be examined g). Subsequently, all emission signals 8 are analysed and evaluated. In a first step h) it is determined whether the emission signals 8 are characteristic of a deposit and thus indicate a deposit on the ocular fundus or a precursor of a drusen. If this is the case, in a second step i) this characteristic emission signal 8 is traced back to the emitting pixel so that the deposit can be localised.

FIG. 2a shows a spectral imaging device 1 for in vivo detection of autofluorescence of an ocular fundus of an eye 2 according to a preferred embodiment of the invention in a schematic view. The spectral imaging device 1 has a light source 3 that is designed to produce a light signal 4 in the form of a laser beam with a wavelength in the blue or deep-blue range. This light signal 4 for stimulating the ocular fundus is filtered by means of a stimulation filter 11 before being passed on to the scanning unit 6. The scanning unit has a plurality of deformable mirrors 10 that can be selectively controlled and moved so that the light signal 4 can be deflected in both the vertical and horizontal directions. The scanning unit 6 is coupled to a position correction unit 5, which permanently monitors the position of the eye and adjusts the mirrors accordingly so that individual virtual background pixels that move due to eye movement can be controlled. The emission signals 8 emitted by the ocular fundus are first filtered by an emission filter 12 and then detected by a sensor 7. The evaluation unit 9 is coupled to the sensor 7 so that the emission signals 8 can be forwarded directly to the evaluation unit 9. The evaluation unit 9 then detects deposits on the ocular fundus or precursors of drusen and localises them according to the tracked pixels.

FIG. 2b shows a schematic histological representation of deposits in an ocular fundus in a vertical sectional view. In particular, small deposits can be visualised using the imaging device described above. The imaging device will produce a frontal (en face) image. Four structural layers are shown: retinal pigment epithelium I, Bruch's membrane II, choriocapillaris III, and choroid IV. Sub-RPE deposits can be detected in the area between Bruch's membrane II and the RPE I. Not only smaller deposits, such as low-risk A drusen, can be visualised. Rather, the method and spectral imaging device 1 according to the invention also make it possible to detect micro-drusen B as well as basal linear deposits and their precursors (pre-BLinD, BLinD) C, which can be easily distinguished from surrounding structures in the overview microscopy.

### LIST OF REFERENCES

1 Spectral imaging device
2 Eye
3 Light source
4 Light signal
5 Position correction unit
6 Scanning unit
7 Sensor
8 Emission signal
9 Evaluation unit
10 Mirror
11 Excitation filter
12 Emission filter
13 Image
I Retinal pigment epithelium (RPE)
II Bruch's membrane
III Choriocapillaris
IV Choroid
A Hard low-risk drusen
B Microdrusen
C BLinD

1. A method for in vivo detection of autofluorescence of an ocular fundus of an eye the method comprising the following method steps:
a) providing a light signal with a predetermined wavelength in a short-wave range;
b) detecting a first eye position and dividing the ocular fundus into a grid comprising a multiplicity of pixels depending on the detected first eye position;
c) determining an area of the ocular fundus to be examined, wherein the area of the ocular fundus to be examined is determined by a selection of a plurality of pixels;
d1) exciting the ocular fundus of a pixel of the area to be examined using the light signal;
d2) detecting a further ocular position differing from the first ocular position and tracking the grid in accordance with the detected further ocular position,
e) detecting at least one emission signal emitted by the pixel of the ocular fundus;
f) supplying the emission signal to an evaluation unit; and
g) repeating steps d1) to f) for each pixel of the area to be examined.

2. The method according to claim 1, further comprising:
h) determining, on the basis of the emission signal, whether a deposit is present on the ocular fundus; and
i) assigning the deposit to a pixel.

3. The method according to claim 1, wherein the predetermined wavelength can be selected from a range between 350 nm and 750 nm.

4. The method according to claim 1, wherein the predetermined wavelength can be continuously adjusted.

5. A spectral imaging device for in vivo detection of autofluorescence of an ocular fundus of an eye, the spectral imaging device comprising:
a light source for generating a light signal with a predetermined wavelength in a short-wave range for exciting at least one region of the ocular fundus;
a position correction unit with a tracking system for detecting and tracking an eye position;
a scanning unit for scanning at least one area of the ocular fundus depending on the position of the eye; and
a sensor for detecting an emission signal emitted by the ocular fundus depending on the position of the eye.

6. The spectral imaging device according to claim 5,
wherein the light source is configured to generate a light signal comprising a predetermined wavelength in a range between 350 nm and 750 nm.

7. The spectral imaging device according to claim 5, wherein the scanning unit comprises a plurality of deformable mirrors.

8. The spectral imaging device according to claim 5, further comprising an evaluation unit for determining, on the basis of the emission signal, whether a deposit is present on the ocular fundus, and for localising the deposit based on the position of the eye.

9. The spectral imaging device according to claim 5, wherein the light source comprises an excitation filter for filtering the light signal.

10. The spectral imaging device according to claim 5, wherein the sensor comprises an emission filter for filtering the emission signal.
